(19) United States
(12) Patent Application Publication
(10) Pub. No.: US 2017/0007958 A1
(43) Pub. Date: Jan. 12, 2017

(54) Gas Processing Apparatus

(71) Applicant: KABUSHIKI KAISHA TOSHIBA, Tokyo (JP)

(72) Inventors: Akio UI, Tokyo (JP); Yosuke SATO, Kawasaki (JP); Masato AKITA, Kawasaki (JP)

(73) Assignee: Kabushiki Kaisha Toshiba

(21) Appl. No.: 15/204,229

(22) Filed: Jul. 7, 2016

(30) Foreign Application Priority Data
Jul. 10, 2015 (JP) 2015-139078

(51) Int. Cl. B01D 53/32; B01D 53/34; B01D 53/86; A61L 9/16

(52) U.S. Cl. CPC B01D 53/32; A61L 9/16; B01D 53/346; B01D 53/8675; A61L 2209/111; B01D 2259/818; B01D 2257/106

(57) ABSTRACT

A gas processing apparatus of an embodiment has stacks, gas flow paths, an AC power supply, and a flow limiter. The stacks are away from each other and in parallel. Each stack includes a dielectric substrate and a first to a third electrode. The first and second electrodes are respectively disposed on the first and second main surfaces of the dielectric substrate. The third electrode is disposed inside the dielectric substrates. The gas flow paths supply a target gas between the stacks, The AC power supply applies an AC voltage across the first and second electrodes and the third electrodes, so as to generate plasma induced flows of the target gas between the dielectric substrates. The flow limiter is disposed on a downstream side of the stacks and limits a flow rate of the target gas.

### CROSS REFERENCE TO RELATED APPLICATION

This application is based upon and claims the benefit of priority from Japanese Patent Application No. 2015-139078, filed on Jul. 10, 2015; the entire contents of which are incorporated herein by reference.

### FIELD

Embodiments described herein relate generally to a gas processing apparatus.

### BACKGROUND

Atmospheric gas in a living space, a refrigerator, a warehouse, or the like or exhaust gas from a processing apparatus may include toxic substances, malodorous substances, and/or the like. Small, high-efficiency gas decomposition apparatuses (including air purifiers, air purifying air-conditioners, and gas purifiers) are demanded for decomposing, sterilizing, and/or the like (hereinafter referred to as gas decomposition) of such toxic substances, malodorous substances, and/or the like.

In general, in a gas decomposition apparatus, atmospheric air or process exhaust gas including a target gas (gas as a target of decomposition) is introduced into a gas decomposition chamber by a blower, The target gas introduced into the gas decomposition chamber is decomposed and purified by electric discharge, decomposition catalyst, photocatalyst, radicals of ozone or the like generated by electric discharge.

It is not always easy to efficiently process gas in the gas decomposition apparatus.

### BRIEF DESCRIPTION OF THE DRAWINGS

FIG. 1 is a side view illustrating an overall structure of a gas decomposition apparatus 10 according to a first embodiment.

FIG. 2 is an enlarged schematic view representing details of gas decomposition elements 20 constituting a processing unit U.

FIG. 3 is an enlarged side view conceptually representing an operating state of the gas decomposition elements 20.

FIG. 4 is a graph representing a distribution of flow velocity V of the plasma induced flow Fp.

FIG. 5 is a graph representing dependency of the applied voltage Vrf on the maximum flow velocity Vmax of the plasma induced flow Fp.

FIG. 6 is a side view representing an overall structure of a gas decomposition apparatus 10x according to a comparative example.

FIG. 7 is a graph representing test results of a toluene gas decomposition experiment using the processing unit U.

FIG. 8 is a side view illustrating the overall structure of a gas decomposition apparatus 10a according to a second embodiment.

FIG. 9 is a side view illustrating the overall structure of a gas decomposition apparatus 10b according to a third embodiment.

FIG. 10 is a side view illustrating the structure of a processing unit Ub of the gas decomposition. apparatus 10b.

FIG. 11 is a side view illustrating the overall structure of a gas decomposition apparatus 10c according to a fourth embodiment.

FIG. 12 is a side view illustrating the structure of a processing unit Ud of a gas decomposition apparatus 10d according to a fifth embodiment.

FIG. 13 is a side view illustrating the structure of a processing unit Uy of a gas decomposition apparatus 10y according to a comparative example 2.

FIG. 14 is a side view illustrating the structure of a processing unit Ue of a gas decomposition apparatus 10e according to a sixth embodiment.

FIG. 15 is a side view illustrating the structure of a gas decomposition apparatus 10e according to a modification example of the sixth embodiment.

### DETAILED DESCRIPTION

In one embodiment, a gas processing apparatus includes a plurality of stacks, a plurality of gas flow paths, an AC power supply, and a flow limiter. The stacks are away from each other and in parallel. Each stack includes a dielectric substrate and a first to a third electrode, The dielectric substrate has a first and a second main surface, The first and second electrodes are respectively disposed on the first and second main surfaces. The third electrode is disposed inside the dielectric substrates. The gas flow paths supply a target gas between the stacks. The AC power supply applies an AC voltage across the first and second electrodes and the third electrodes, so as to generate plasma induced flows of the target gas between the dielectric substrates. The flow limiter is disposed on a downstream side of the stacks and limits a flow rate of the target gas.

Hereinafter, embodiments will be explained in detail with reference to drawings.

### First Embodiment

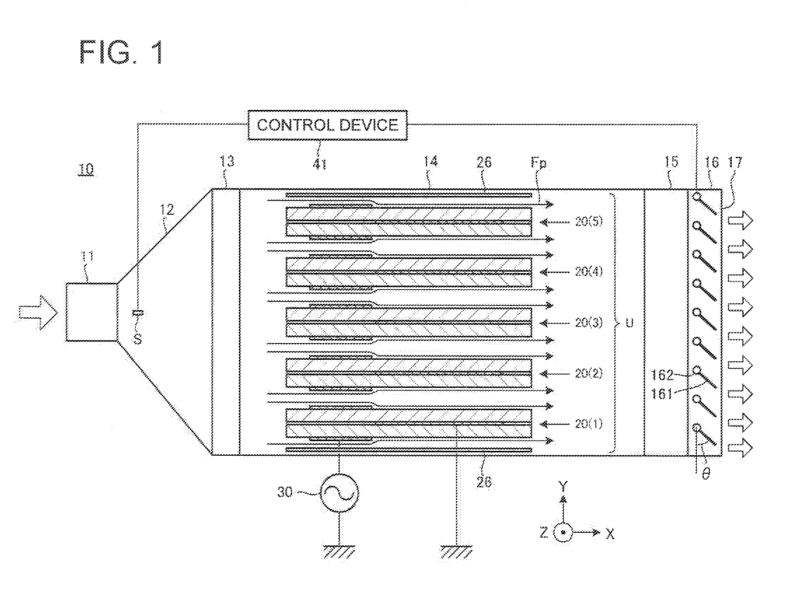

FIG. 1 illustrates an overall structure of a gas decomposition apparatus 10 according to a first embodiment.

The gas decomposition apparatus 10 decomposes a target vas (gas containing at least one of carbon or nitride, for example, formaldehyde, toluene, acetaldehyde, ammonia gas) contained in atmosphere or process exhaust gas by electric discharge generated by a high AC voltage applied across discharge electrodes and ground electrodes. The gas decomposition apparatus 10 functions as a gas processing apparatus which processes a gas to be processed.

The gas decomposition apparatus 10 has a gas introduction port 11, a flow path expansion chamber 12, a prefilter 13, a gas decomposition chamber 14, an ozone treatment chamber 15, and a flow adjuster 16, and insides thereof are gas flow spaces.

Through the gas introduction port 11, the gas to be processed including the target gas is introduced.

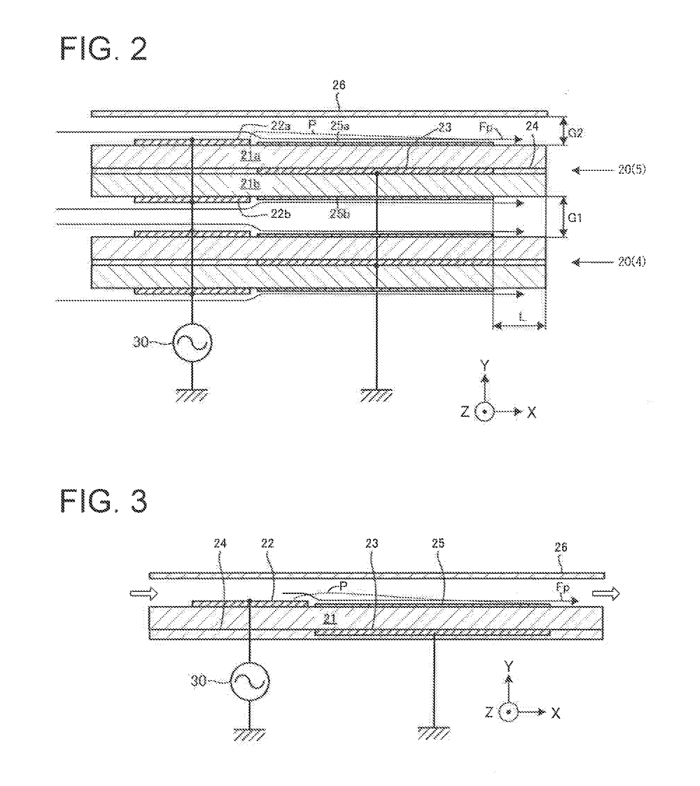

The flow path expansion chamber 12 expands the flow path from the gas introduction port 11 to the prefilter 13 and the gas decomposition chamber 14. The flow path expansion chamber 12 is a gas flow path supplying the gas to be processed to spaces among discharge electrodes 22 (or spaces between the discharge electrodes 22 and gas flow partitions 26) which are illustrated in following FIG. 2.

The prefilter 13 removes dust, dirt, and so on in the gas flowing into the gas decomposition chamber 14.

In the gas decomposition chamber 14, a processing unit U including a plurality of gas decomposition elements 20(1) to 20(5) is disposed, so as to process the gas to be processed. Note that details of the processing unit U will be described later.

The ozone treatment chamber 15 has an ozone processor (not illustrated, for example an ozone catalyst), and processes and decomposes high-concentration ozone gas, NOx, or SOx generated in the gas decomposition chamber 14.

The flow adjuster 16 is disposed on a downstream side of the ozone treatment chamber 15 and limits the flow rate of the gas to be processed. By limiting the flow rate of the gas to be processed, the gas to be processed is supplied at an appropriate flow rate to the processing unit U, so as to improve gas decomposition efficiency in the processing unit U. The flow adjuster 16 is disposed on a downstream side of a plurality of stacks and functions as a flow limiter limiting the flow rate of the target gas.

Here, the flow adjuster 16 uses a louver constituted of a plurality of flow limiting plates (plates) 161. The flow limiting plates 161 are disposed vertically and can. be inclined about respective rotation axes 162, so as to limit the flow rate of the gas to be processed.

The rotation axes 162 are disposed on respective upper ends of the flow limiting plates 161, but the rotation axes 162 may be disposed on respective lower ends or middle positions (center positions) of the flow limiting plates 161.

The angle θ which the flow limiting plates 161 form in the vertical direction can be changed to adjust the flow rate of the gas to be processed. When the angle θ is zero, the upper and lower ends of the flow limiting plates 161 are substantially in contact, and the flow adjuster 16 is closed (the flow rate of the gas to be processed becomes substantially zero). When the angle θ is 90°, the direction of the flow limiting plates 161 is in a state of being along the flow of the gas to be processed, and the flow adjuster 16 is open the flow adjuster 16 barely limits the flow rate of the gas to be processed).

In order to make the flow limiting plates 161 operate simultaneously, the flow limiting plates 161 can be connected. For example, when lower ends of the flow limiting plates 161 are connected with a rod-shaped body, vertically pushing or pulling the rod-shaped body can change the angle θ of the flow limiting plates 161 at once.

Further, in order to change the inclination of the flow limiting plates 161, a driving device (a motor for example) can be used. The driving device functions as a regulator regulating the angle of the plates. This driving device may be provided for each of the flow limiting plates 161, or may be configured to move the rod-shaped body when the flow limiting plates 161 are connected with the rod-shaped body.

Here, the louver is used as the mechanism of the flow adjuster 16, but another mechanism, for example, a valve (valve mechanism) or a blower can be employed. By opening or closing the valve, the flow rate of the gas to be processed can be adjusted. By changing the direction of blow and the rate of blow by the blower, the flow rate of the gas to be processed can be adjusted. For example, the direction of blow by the blower can be set reverse to the direction of flow of the gas to be processed to make the rate of blow correspond to the introduction amount of the gas to be processed from the gas introduction. port 11 making them counterbalance each other, thereby stopping or limiting the introduction of the gas to be processed through the gas introduction port 11. Further, the direction of blow by the blower can be made the same as the direction of flow of the gas to be processed to make the rate of blow small or substantially zero, so as to limit the flow rate of the gas to be processed.

Through a gas outlet 17, the gas to be processed including the decomposed target gas flows out.

The gas to be processed is introduced through the gas introduction port 11, passes through the prefilter 13, the gas decomposition chamber 14 (processing unit U), and the ozone treatment chamber 15, and is exhausted through the gas outlet 17.

A detector S detects the flow rate of the gas to be processed.

A control device 41 controls the flow adjuster 16 according to a result of detection by the detector S, and so on, so as to limit the flow rate of the gas to be processed.

FIG. 2 represents details of gas decomposition elements (plasma actuators using Dielectric Barrier Discharge (DBD)) 20 constituting the processing unit U in magnification.

The processing unit U has gas decomposition elements 20 (20(1) to 20(5)) and gas flow partitions 26. Here, the number of gas decomposition elements 20 included in the processing unit U is five, but it can be changed appropriately.

The gas decomposition elements 20 have dielectric substrates 21 (21a, 21b), discharge electrodes 22 (22a, 22b), ground electrodes 23, insulating sealing layers 24, and photocatalyst layers 25 (25a, 25b).

The dielectric substrates 21a, 21b, the discharge electrodes 22a, 22b (first, second electrodes), the ground electrode 23 (third electrode), the insulating sealing layer 24, and the photocatalyst layers 25a, 25b of one gas decomposition element 20 function as a stack.

The dielectric substrates 21 are substrates of dielectric material (for example, quartz, silicon rubber, or kapton (one kind of polyimide)). For example, quartz plates with a thickness of 1 mm can be used as the dielectric substrates 21.

The discharge electrodes 22 and the ground electrode 23 are constituted of a conductor of metal or the like. For example, a gold (Au) thin film can be formed on the dielectric substrates 21 by means of sputtering or plating, so as to make the discharge electrodes 22 and the ground electrode 23.

The adjacent dielectric substrates 21 of gas decomposition elements 20 are disposed to have a gap G1.

The dielectric substrates 21 of the top (and bottom) gas decomposition element 20 are disposed with a gap G2 from the gas flow partition 26. Note that here the thicknesses of the discharge electrodes 22 and the thicknesses of the photocatalyst layers 25 can be ignored with respect to the gaps G1, G2.

The discharge electrodes 22 (22a, 22b), the ground electrode 23, and the dielectric substrates 21 (21a, 21b) are in the sizes of, for example, 2 mm×30 mm, 10 mm×30 mm, and 20 mm×50 mm, respectively, in longitudinal direction (X direction) and depth (Z direction). Further, the gap G (G1, G2) is 2 mm for example. Consequently, the gas decomposition chamber 14 (processing unit U) is made compact in the size of, for example, 20 mm×30 mm×50 mm in X, Y, and Z direction.

The insulating sealing layer 24 is a dielectric film for preventing reverse discharge in the ground electrode 23. For example, a silicon oxide film, an induced insulating film, an insulating silicone filler, or a kapton tape coating can be used as the insulating sealing layer 24.

When there is a space around the ground electrode 23, a reverse flow caused by reverse discharge may hinder a plasma induced flow Fp, which will be described later, or abnormal discharge (overheating) may occur in a small space. In order to prevent them, it is desired to tightly seal the vicinity of the insulating sealing layer 24 with the insulating sealing layer 24.

The photocatalyst layers 25a, 25b are layers of photocatalyst material (TiO₂ for example), and are disposed in the vicinity of plasma P or in plasma P on the dielectric substrates 21. The photocatalyst layers 25a, 25b may be formed by, for example, coating a photocatalyst material.

The photocatalyst layers 25a, 25b are activated by light emission from the plasma P, and remove NOx or the like contained in the plasma P. That is, the as decomposition rate can be improved by decomposition of gas by the plasma P itself and the photocatalysts operating together.

Note that the gas decomposition elements 20 need not have the photocatalyst layers 25. However, when the gas decomposition elements 20 have the photocatalyst layers 25, decomposition of gas can be accelerated further.

A high-voltage AC power supply 30 applies a high AC voltage (for example, sinusoidal voltage of 10 kHz and 6 kV) between the discharge electrodes 22a, 22b and the ground electrode 23.

Here, the adjacent dielectric substrates 21 are disposed so as to oppose each other with the gap G1.

Further, the gas flow partitions 26 are disposed so as to oppose the dielectric substrates 21 at the top layer and the bottom layer with the gap G2. The gas flow partitions 26 are partition walls limiting the flow path of the gas to be processed. The gas flow partitions 26 limit the gas to be processed passing through a top part and a bottom part of the processing unit U so that the gas to be processed passes through the plasma P and does not pass through the outside of the plasma P.

The gap G1 is, as will be described later, equal to or less than 1.3 times the total value of thicknesses h of the plasma induced flow Fp over the pair of opposing dielectric substrates 21 (G1≦1.3*2 h=2.6 h). Specifically, the gap G1 is preferably 2 mm or more and 8 mm or less. In order to increase the decomposition efficiency of hardly decomposable gas, more preferably, the gap G1 is 2 mm or more and 6 mm or less to satisfy “G1≦1.0*h”.

The gap G2 is equal to or less than 1.3 times the thickness h of the plasma induced flow Fp over the dielectric substrate 21 (G2≦1.3*h). Specifically, the gap G2 is preferably 1 mm or more and 4 mm or less. In order to increase the decomposition rate of hardly decomposable gas, more preferably, the gap G2 is 1 mm or more and 3 mm or less to satisfy “G2≦1.0*h”.

By thus setting the gaps G1, G2, active oxygen (OH radicals and O radicals) in the plasma P is effectively utilized, so as to efficiently decompose the target gas.

In this manner, by making the gaps G1, G2 narrow, all decomposed gases pass through the inside or vicinity of the plasma P (prevention of passing by), thereby improving the gas decomposition rate.

FIG. 3 schematically represents an operating state of the gas decomposition elements 20.

By the high AC voltage from the high-voltage AC power supply 30, the plasma P is generated on the surface of the dielectric substrate 21 in the ground electrode 23 direction from the discharge electrode 22. The plasma P includes positive ions and electrons. The positive ions flow from the discharge electrodes 22 on the surface of the dielectric substrates 21 above the ground electrode 23. This flow collides with an atmosphere and accompanies a gas flow around it, thereby generating the plasma induced flow Fp.

Electrons accumulate on the surface of the dielectric substrates 21 in contact with the plasma P, thereby charging up to be negative. Accordingly, in the surface direction of the dielectric substrates 21 corresponding to the direction from the discharge electrodes 22 to the ground electrode 23, positive ions flow evenly. Since the generated plasma P is thin surface plasma, the plasma induced flow Fp also becomes a surface flow flowing in the vicinity of the dielectric substrates 21. Specifically, disposing the ground electrode 23 by displacing in the X direction relative to the discharge electrodes 22 allows generating the plasma induced flow Fp flowing in the X direction is generated.

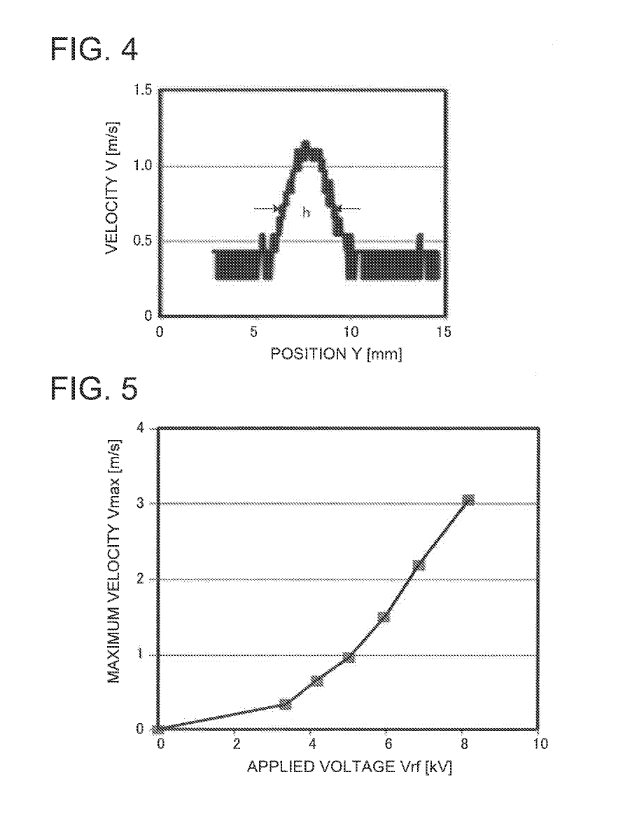

FIG. 4 is a graph representing a distribution of flow velocity V of the plasma induced flow Fp.

The horizontal axis is the position Y in the vertical direction of the dielectric substrates 21, and the position of Y=6 mm corresponds to the surface of the dielectric substrates 21. A hot-wire air probe of the flow meter was moved vertically, so as to measure the distribution of the flow velocity V of the plasma induced flow Fp. Here, a sinusoidal voltage with a frequency f of 10 kHz and an applied voltage Vrf of 6 kV is applied.

As illustrated in FIG. 4, the plasma induced flow Fp flows only in the vicinity of the surfaces (half value width (thickness) h=2.4 mm) of the dielectric substrates 21. That is, the plasma induced flow Fp with a thickness h of 2.4 mm occurs.

FIG. 5 is a graph representing dependency of the applied voltage Vrf of the maximum flow velocity Vmax of the plasma induced flow Fp. As illustrated in FIG. 5, the plasma induced flow Fp is generated from the applied voltage Vrf of about 3 kV. As the applied voltage Vrf increases, the density (ion density) of the plasma becomes high and an attracting electric field also becomes large. Accordingly, as the applied voltage Vrf increases, the flow velocity V of the plasma induced flow Fp increases.

Without generating a flow (pressure) from the outside by using the blower such as a fan, the plasma induced flow Fp is generated on the discharge electrodes 22. As already described, the gaps G1, G2 are small. Accordingly, the gas to be processed sucked in from an upstream side passes through the plasma P.

The frequency f and the applied voltage Vrf can he changed appropriately in the range of 2 kHz to 200 kHz and in the range of 3 kV to 15 kV, respectively. Particularly preferred ranges of the frequency f and the applied voltage Vrf are 5 kHz to 60 kHz and 3.5 kV to 10 kV, respectively. When the frequency f and the applied voltage Vrf are changed in this manner, the thickness h of the plasma induced flow Fp varies in the range of 1.5 mm to 3 mm. Consequently, the gap G1 and the gap G2 can be set appropriately in, for example, the range of 2 mm to 8 mm and the range of 1 mm to 4 mm, respectively.

Next, the gas decomposition mechanism will he described. The atmosphere and the target gas are partially decomposed by discharge.

From oxygen gas (O₂) and moisture (H₂O) in the atmosphere and hydrogen, nitrogen, and oxygen components in the target gas, active oxygen (hydroxyl radicals: OH, oxygen atom radicals: O, ozone: O₃, and/or the like) are generated.

Then, when the target gas (for example, formaldehyde, toluene, acetaldehyde, or ammonia gas) is mixed with an oxidant, it is decomposed into CO₂, H₂O, NOx (NO₂, NO), and so on by reactions represented by (1), (2), (3). Note that the NOx can be removed by photocatalyst layers 25a, 25b and the ozone treatment chamber 15 disposed in the later stage.

Target gas+OH→CO₂+H₂O+NOx (1)

Target gas+O→CO₂+H₂O+NOx (2)

Target gas+O₃→CO₂+H₂O +NOx (3)

As described above, in this embodiment, the flow adjuster 16 limits the flow rate of the gas to be processed, thereby improving efficiency of decomposition of gas by the processing unit U.

### Comparative Example

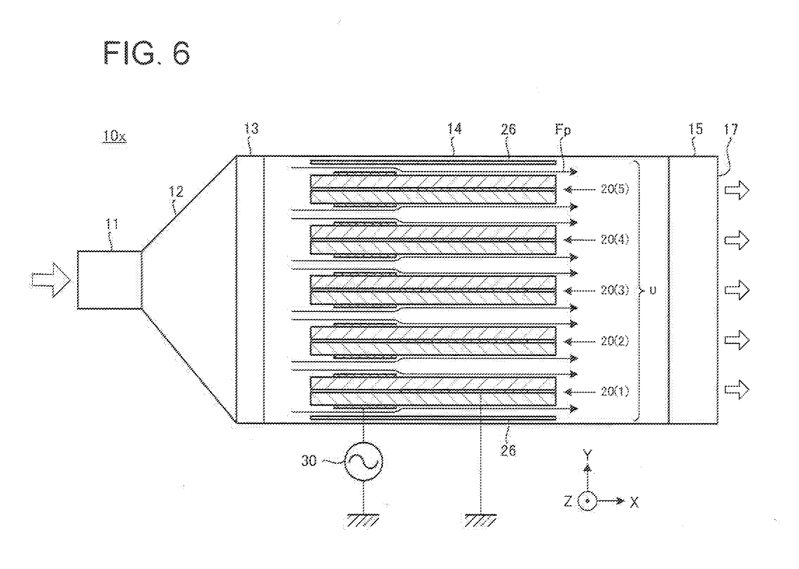

FIG. 6 illustrates an overall structure of a gas decomposition apparatus 10x.

according to a comparative example. The gas decomposition apparatus 10x does not have the flow adjuster 16, Accordingly, the flow rate of the gas to be processed flowing into the processing unit U becomes too large, and the efficiency of gas decomposition tends to decrease.

In detail, by increase of the flow rate or the concentration of the gas to be processed flowing into the processing unit U, the following problems (1) to (3) occur,

### (1) Decrease in Decomposition Efficiency

### (2) Decrease in Complete Oxidation Rate

### (3) Occurrence of Dirt on the Discharge Electrodes 22

Dirt on the discharge electrodes 22 can also cause further decrease in decomposition efficiency and instability of discharge.

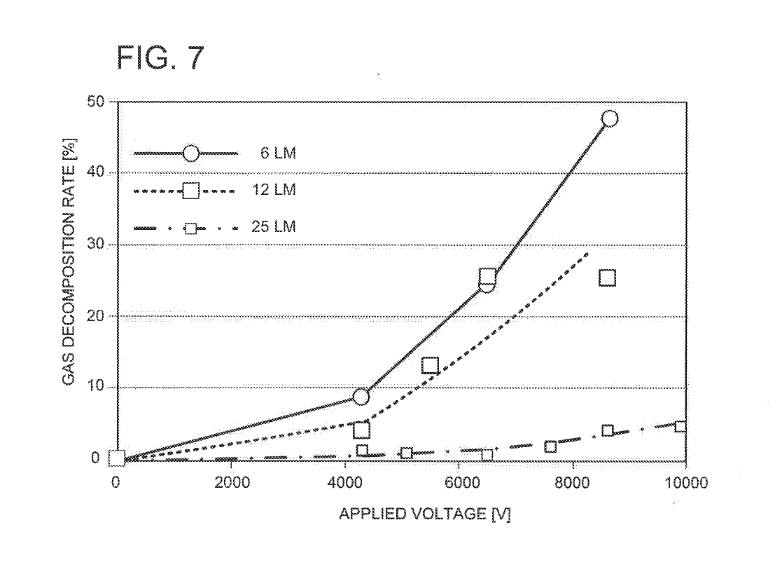

The applied voltage of the gas decomposition efficiency and the flow rate dependency will be explained. FIG. 7 illustrates test results of a toluene gas decomposition experiment using the processing unit U A test gas containing toluene gas was passed through the processing unit U, and the concentration of the toluene gas and the gas decomposition rate (%) after the passing were measured.

Here, the voltage to be applied to the processing unit U was 9500 V. The toluene gas concentration in the test gas was about 50 ppm. For measurement of the toluene gas concentration and the gas decomposition rate (%), an FT-IR apparatus was used.

The horizontal axis of FIG. 7 represents the applied voltage and the vertical axis represents the toluene gas decomposition rate, It can be seen that as the applied voltage increases, the plasma intensifies and the decomposition rate increases.

Here, as the flow rate of the test gas increases to 6, 12, 25 LM (L/min), the decomposition rate decreases significantly to about 50%, 30%, 5%. This is because the amount of generating highly active OH radicals generated in the plasma P is infinite and does not catch up with the amount of toluene supplied.

A complete oxidation and decomposition reaction (toluene deodorizing reaction) of OH of toluene is represented by expression (4).

C₆H₅CH₃+18OH→7CO₂+4H₂O +5H₂ (4)

Note that besides the product (CO₂ or the like) on the right side of expression (4), it is possible that CO, CH₃CHO, CH₃COOH occur as by-products.

In order to completely oxidize toluene, for 50 ppm toluene, the number of OH molecules that is 18 times larger, that is, 900 ppm of OH molecules, is necessary. The OH radicals have large reaction rate, but when the amount of OH is not sufficient, incomplete combustion reaction may occur or undecomposed toluene may remain.

Note that when the concentration of the toluene gas increases, similarly, incomplete combustion reaction and/or undecomposed toluene occurs.

The atmosphere normally contains about 20% of O₂ and 1% to few % H₂O, and is usable as a raw material for OH generation.

Here, the magnitude relation between the rate of generation of OH radicals from O₂ and H₂O in the plasma P of the atmosphere and the rate of reaction of OH and toluene is important for progress of the decomposition reaction of toluene.

On the other hand, the life span of OH radicals is about 700 μ seconds. The flow velocity in the processing unit U is 1 to 3 m/s. Accordingly, OH radicals generated in the plasma P can exit by only 1 or 2 mm (survive) from the plasma P. That is, in order to let the decomposition reaction of toluene proceed (condition that the plasma is constant), it is necessary to elongate the time for the reaction gas to stay in the plasma P.

The above-described problems ((1) decrease in decomposition efficiency, (2) decrease in complete oxidation rate, and (3) occurrence of dirt on the discharge electrodes 22) are caused by that the residence time of the target gas in the plasma P becomes short due to increase in flow rate or concentration of the process target gas.

In this embodiment, by limiting the flow rate of the gas to be processed by the flow adjuster 16, the problems (1) to (3) can be solved. consequently, decomposition with high efficiency becomes possible with the small gas decomposition apparatus 10.

### Second Embodiment

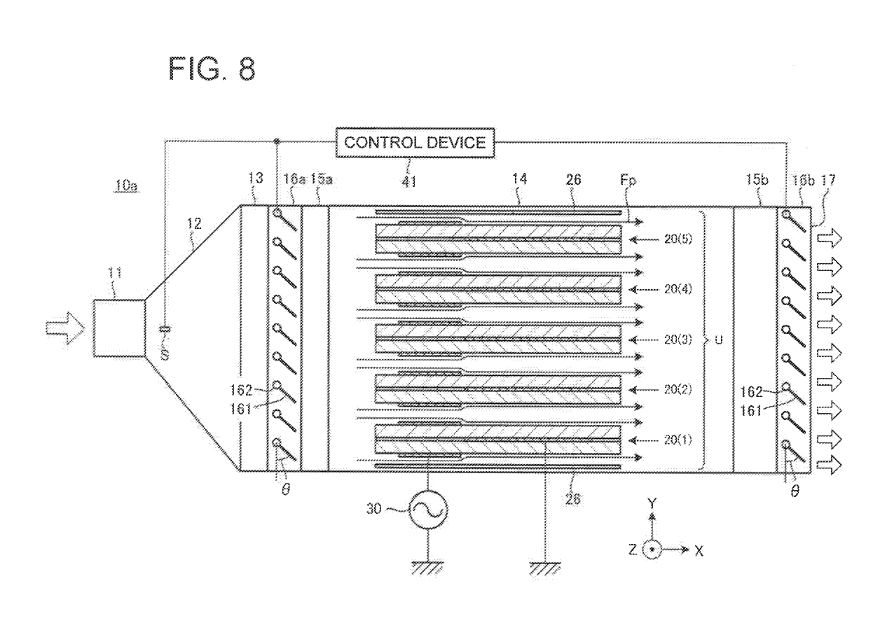

FIG. 8 illustrates the overall structure of a gas decomposition apparatus 10a according to the second embodiment

The gas decomposition apparatus 10a has a gas introduction port 11, a flow path expansion chamber 12, a prefilter 13, a gas decomposition chamber 14, ozone treatment chambers 15a, 15b, flow adjusters 16a, 16b, and a gas outlet 17, and insides thereof are gas flow spaces. The ozone treatment chambers 15a, 15b are disposed on both of an upstream side and a downstream side of the gas decomposition chamber 14. The flow adjusters 16a, 16b are disposed on an upstream side of the ozone treatment chamber 15a and a downstream side of the ozone treatment chamber 15b, respectively.

### Modification examples of the first and second embodiments

The flow adjuster 16 can be used to adjust the flow rate of the gas to be processed, so as to change the residence time.

### (1) Adjustment According to the Gas Type

The degree of opening of the flow adjuster 16 can be adjusted according to the type of the gas to be processed. For example, in the case of hardly decomposable gas (for example, toluene), the degree of opening may be decreased to have a low flow velocity (long residence time) to increase the decomposition rate. In the case of easily decomposable gas (for example, formaldehyde), the degree of opening may be increased to have a high flow velocity (short residence time). In this case, a gas analyzer may be provided as the detector S so as to detect the type or the concentration of the gas. Then, a controller 41 adjusts the degree of opening of the flow adjuster 16 according to the result of this detection

### (2) Adjustment According to the Gas Concentration

The controller 41 decreases the degree of opening to have a low flow velocity (long residence time) to increase the decomposition rate when the gas concentration is high. Additionally dirt on the discharge electrodes 22 is prevented thereby. The controller 41 increases the degree of opening to have a high flow velocity (short residence time) to decrease the decomposition rate when the gas concentration is low. Here, the degree of opening of the flow adjuster 16 may be adjusted in consideration of both the gas type and the gas concentration.

### (3) Regular Adjustment

The controller 41 may regularly close the flow adjuster 16 to perform cleaning. When no gas to be processed flows, the OH radicals can clean the discharge electrodes 22.
The degree of opening is made small regularly to have a low flow velocity (long residence time) to increase the OH radical concentration, so as to clean the discharge electrodes 22. This can restore the decomposition rate. At this time, it is further effective to stop the introduction of the gas to be processed to perform the cleaning.

### (4) Detection of Dirt

Dirt on the discharge electrodes 22 may be detected, and according to the result of this detection, the controller 41 may adjust the degree of opening of the flow adjuster 16. Sensors may he used as follows to detect dirt on the discharge electrodes 22 or on the dielectric substrate 21 downstream the discharge electrode 22. For example, an optical sensor is disposed on an upstream or downstream side of the discharge electrodes 22, so as to measure the light emission intensity of plasma in discharge portions of the discharge electrodes 22. With a decrease in light emission intensity, it can be detected that the discharge electrodes 22 or the perimeter are dirty and their discharge are being weak. Further, an ammeter or a wattmeter may be disposed in the high-voltage AC power supply 30, so as to measure a discharge current or discharge power. With a decrease in discharge current or discharge power, it can he detected that the discharge electrodes 22 or the perimeter are dirty and their discharge being weak. Moreover, a gas sensor may be disposed on a downstream side of the discharge electrode 22, so as to measure the concentration of the target gas. With an increase in concentration of the target gas (decrease in gas decomposition ability by discharge), it can be detected that the discharge electrodes 22 or the perimeter are dirty and their discharge being weak.

### Third Embodiment

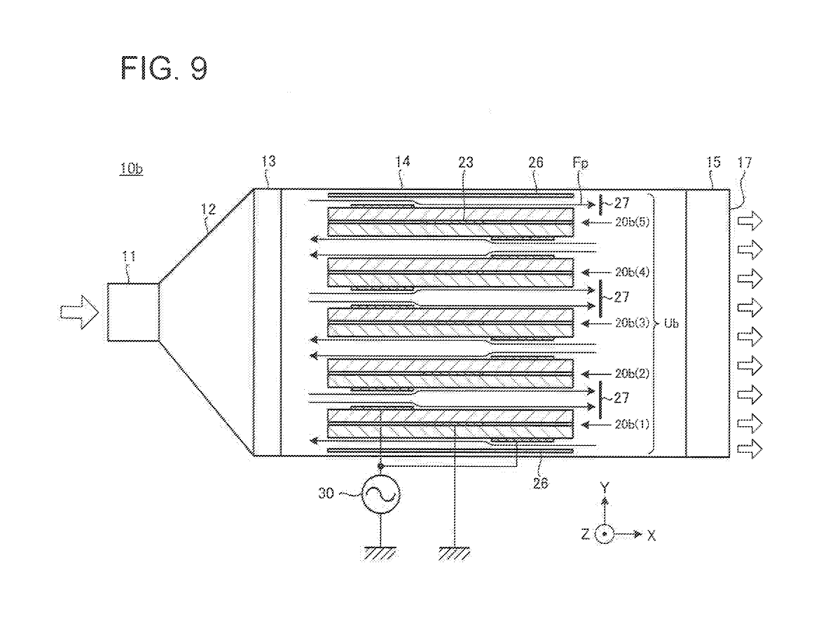

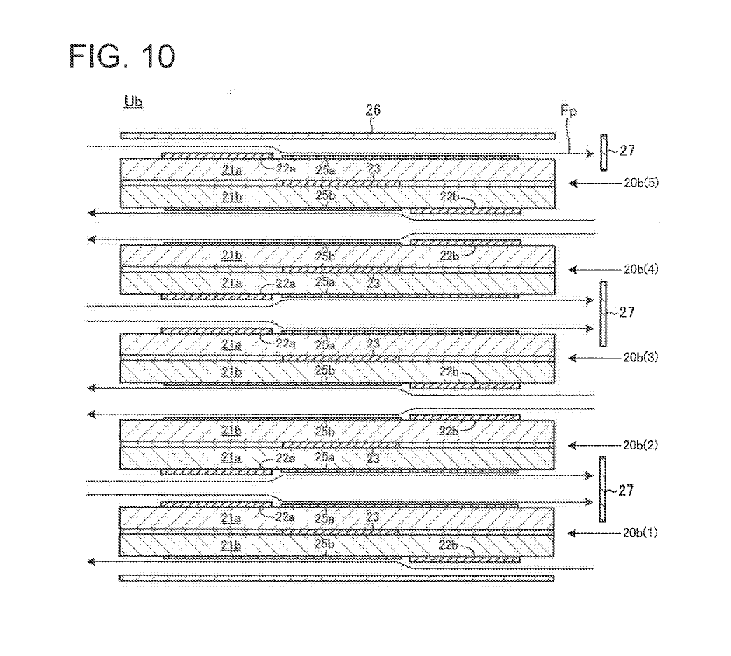

FIG. 9 is a side view illustrating the overall structure of a gas decomposition apparatus lob according to a third embodiment. FIG. 10 is a side view illustrating the structure of a processing unit Ub of the gas decomposition apparatus 10b.

The processing unit Ub has gas decomposition elements 20b(1) to 20b(5), and plasma induced flows Fp flow alternately in opposite directions. That is, plasma induced flows Fp (first plasma induced flows) flow in a rightward direction (forward direction) of the side views between the gas flow partition 26 and the gas decomposition elements 20b(5), between the gas decomposition elements 20b(4), 20b(3), and between the gas decomposition elements 20b(2), 20b(1). Further, the plasma induced flows Fp (second plasma induced flows) flow in a leftward direction (backward direction) of the side views between the gas decomposition elements 20b(5), 20b(4), between the gas decomposition elements 20b(3), 20b(2), and between the gas decomposition element 20b(1) and the gas flow partition 26 (hereinafter referred to as between the gas decomposition elements 20b(5), 20b(4), or the like).

Accordingly, the gas decomposition elements 20b of the processing unit Ub differ from the gas decomposition elements 20 of the processing unit U as follows. Specifically, the discharge electrode 22b and the photocatalyst layer 25b on the dielectric substrate 21b side of the gas decomposition elements 20b are in backward, leftward and rightward directions of the side views to the discharge electrode 22a and the photocatalyst layer 25a on the dielectric substrate 21a side.

Pressure harriers (baffle plates) 27 are disposed so that the plasma induced flows Fp do not easily exit in the forward direction. Accordingly, in the gas decomposition apparatus 10b, as illustrated in FIG. 9, the flow adjuster 16 can he omitted. The pressure barriers (baffle plates) 27 are disposed on the downstream side of the stacks, and function as a flow limiter limiting the flow rate of the target gas. Further, the electrodes 22b between the gas decomposition elements 20b(5), 20b(4) or the like over which the plasma induced flows Fp flow in the leftward direction (backward direction) of the side view are disposed on the downstream side of the gas decomposition elements 20b (stacks), and function as a flow limiter limiting the flow rate of the target gas.

The processing unit Ub can be made compact because of use of the common ground electrodes 23.

### Fourth Embodiment

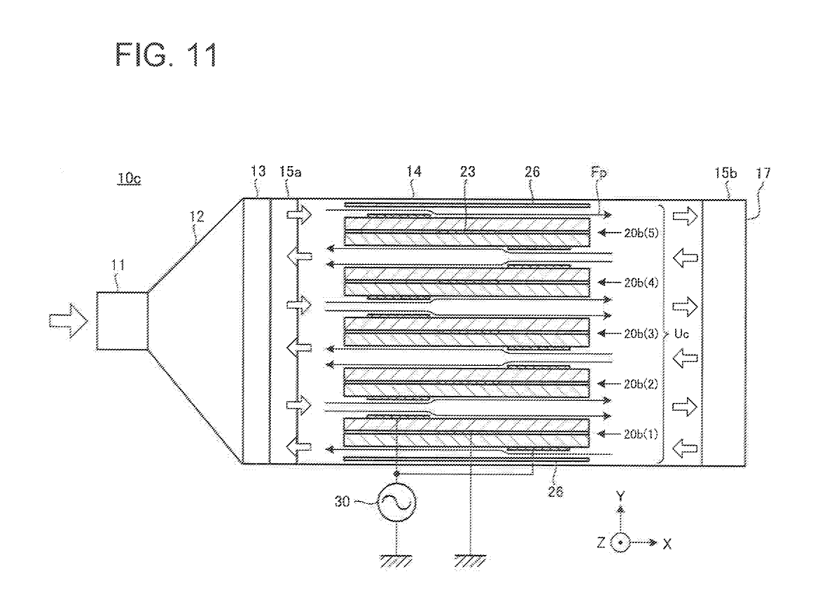

FIG. 11 is a side view illustrating the structure of a processing unit Uc of a gas decomposition apparatus 10c according to a fourth embodiment.

Ozone treatment chambers 15a, 15b (ozone catalysts) are disposed on both upstream and downstream sides of the processing unit Uc.

The processing unit Uc have the same structure as the processing unit Ub except that it does not have the pressure bathers (baffle plates) 27. The gas to be processed flows in or out of the processing unit Uc via both the left and right of the side view of the processing unit Uc.

As already described, the electrodes 22b between the gas decomposition elements 20b(5), 20b(4) or the like over which the plasma induced flows Fp flow in the leftward direction (backward direction) of the side view are disposed on the downstream side of the gas decomposition elements 20b (stacks), and function as the flow limiter limiting the flow rate of the target gas. Consequently, the residence time of the gas to be processed in the processing unit Uc becomes long. Consequently, efficient decomposition of the gas to be processed becomes possible.

### Fifth Embodiment

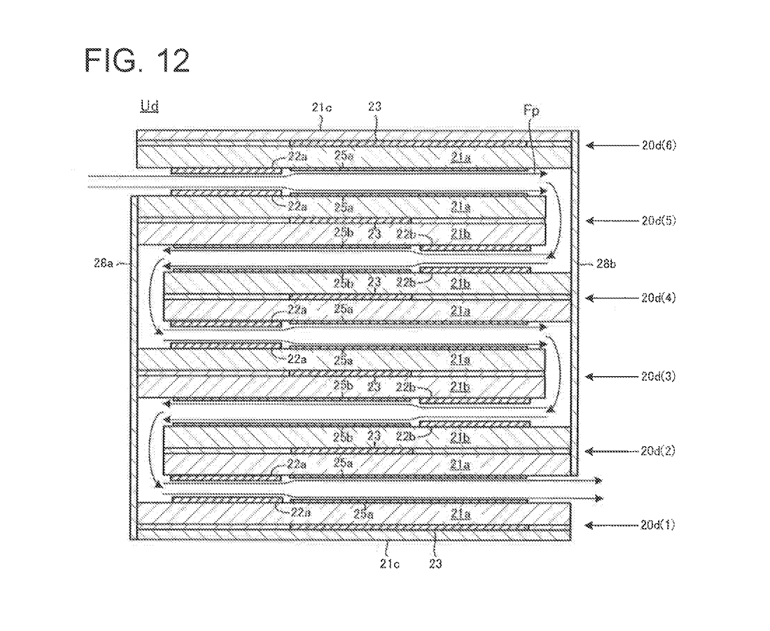

FIG. 12 is a side view illustrating the structure of a processing unit lid of a gas decomposition apparatus 10d according to a fifth embodiment.

The processing unit Ud has gas decomposition elements 20d(1) to 20d(6), and plasma induced flows Fp flow alternately in opposite directions. That is, plasma induced flows Fp flow in a rightward direction (forward direction) of the side view between the gas decomposition elements 20d(6), 20d(5), between the gas decomposition elements 20d(4), 20d(3), and between the gas decomposition elements 20d(2), 20d(1). Further, plasma induced flows Fp flow in a leftward direction (backward direction) of the side view between the gas decomposition elements 20d(5), 20d(4), and between the gas decomposition elements 20d(3), 20d(2) (hereinafter referred to as between the gas decomposition elements 20d(5), 20d(4) or the like).

Here, in order for the plasma. induced flows Fp between the gas decomposition elements 20d(6), 20d(5) to flow into the space between the gas decomposition elements 20d(5), 20d(4), the spaces thereof are connected by a space formed by the gas decomposition elements 20d(6), 20d(4), and a gas flow partition 28b. Further, in. order for the plasma induced flows Fp between the gas decomposition elements 20d(5), 20d(4) to flow into the space between the gas decomposition elements 20d(4), 20d(3), the spaces thereof are connected by a space formed by the gas decomposition elements 20d(5), 20d(3), and a gas flow partition 26a.

That is, the pair of upper and lower gas decomposition elements 20d and the gas flow partition 28 (28a, 28b) function as a guide to guide the first plasma induced flows in the forward direction which exited from the processing unit Ud (plurality of stacks) to the second plasma-induced flows in the backward direction.

Hereinafter, similarly, the plasma induced flows Fp between the gas decomposition elements 20d(4), 20d(3) flow into the space between the gas decomposition elements 20d(3), 20d(2), and the plasma induced flows Fp between the gas decomposition elements 20d(3), 20d(2) flow into the space between the gas decomposition elements 20d(2), 20d(1).

Thus, the gas to be processed which flowed into the space between the gas decomposition elements 20d(6), 20d(5) from the upstream side of the processing unit Ud. pass through the space between the gas decomposition elements 20d(5), 20d(4), the space between the gas decomposition elements 20d(4), 20d(3), the space between the gas decomposition elements 20d(3), 20d(2), and the space between the gas decomposition elements 20d(2), 20d(1), and flow out to the downstream side of the processing unit

The electrodes 22b between the gas decomposition elements 20d(5), 20d(4) or the like over which the plasma induced flows Pp flow in the leftward direction (backward direction) of the side view and the gas flow partition 28b are disposed on the downstream side of the gas decomposition elements 20d (stacks), and function as a flow limiter limiting the flow rate of the target gas.

Note that on the gas decomposition elements 20d(1), 20d(6) on the highest and lowest ends of the processing unit Ud, the discharge electrodes 22a and the photocatalyst layers 25a are disposed only on one side. On the dielectric substrates 21c on the opposite sides thereof, the discharge electrodes 22 and the photocatalyst layers 25 are not disposed. Consequently, it becomes possible to omit the gas flow partitions 26.

Thus, the gas to be processed move in zigzags in the processing unit Ud, and thus the residence time of the gas to be processed in the processing unit Ud becomes long.

That is, by making the flow paths long, the residence time becomes long. Consequently, efficient decomposition of the gas to be processed becomes possible.

### Comparative Example 2

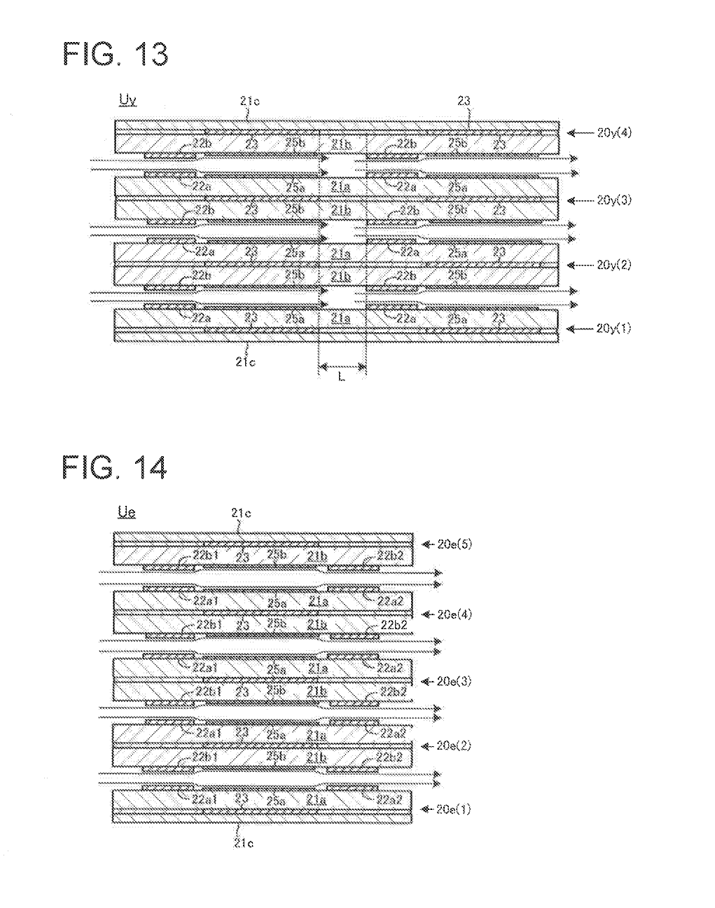

FIG. 13 is a side view illustrating the structure of a processing unit Uy of a gas decomposition apparatus 10y according to comparative example 2.

The processing unit Uy has a structure in which the two processing units U are connected along the flow direction. That is, on the dielectric substrates 21a of the gas decomposition elements 20y, two groups of discharge electrodes 22a and photocatalyst layers 25a are disposed with a gap L. Similarly, on the dielectric substrates 21b, two groups of discharge electrodes 22h and photocatalyst layers 25b are disposed with a gap L.

By disposing the two groups of discharge electrodes 22 and photocatalyst layers 25 in this manner, decomposition efficiency of gas can be improved. However, on the other hand, in order to avoid reverse discharge, it is necessary to have the gap L of a certain length, and it is difficult to make the processing unit Uy compact.

On the other hand, the processing unit Ud of the gas decomposition apparatus 10d according to the fifth embodiment does not need the gap L, and the ground electrodes 23 are shared by the discharge electrodes 22a, 22b. Thus, the processing unit Ud can be made compact.

### Sixth Embodiment

FIG. 14 is a side view illustrating the structure of a processing unit Lie of a gas decomposition apparatus 10e according to a sixth embodiment.

Gas decomposition elements 20e(1) to 20e(5) of the processing unit Ue have two groups of discharge electrodes 22a (22a1, 22a2 (first, fourth electrodes)) and discharge electrodes 22h (22b1, 22b2 (second, fifth electrodes)) on each of their upper side and lower side.

Thus, flow paths can be changed by high-voltage application to the discharge electrodes 22a1, 22a2 and the discharge electrodes 22b1, 22b2 and change of grounding. For example, when a voltage is applied to the discharge electrodes 22a1, 22b1 and the voltage is not applied to the discharge electrodes 22a2, 22b2, the plasma induced flows Fp flow rightward from the left of the side view as illustrated in FIG. 14. On the other hand, when the voltage is applied to the discharge electrodes 22a2, 22b2 and the voltage is not applied to the discharge electrodes 22a1, 22b1, the plasma induced flows Fp flow leftward from the right of the side view (not illustrated).

Specifically, a switch can be used to switch the discharge electrodes 22a1, 22a2 (first and second electrodes) and the discharge electrodes 22b1, 22b2 (fourth and fifth electrodes) to apply the AC voltage from the high-voltage AC power supply 30, thereby switching the direction in which the plasma induced flows Fp flow.

At this time, the discharge electrodes 22a2, 22b2 over which the plasma induced flows Fp flow in the leftward direction (backward direction) of the side view when the voltage is applied are disposed on the downstream side of the gas decomposition elements 20e (stacks), and function as a flow limiter limiting the flow rate of the target gas.

In this manner, the flow path and the residence time of the gas to be processed can be changed in the gas decomposition chamber 14.

Here, the flow path and the residence time may be changed according to the gas type and the amount of decomposition. For example, in the case of easily decomposable gas (for example, formaldehyde), it is conceivable that the voltage is applied to the discharge electrodes 22a1, 22b1 of the gas decomposition elements 20e(1) to 20e(5), and the voltage is not applied to the discharge electrodes 22a2, 22b2. In this case, the plasma induced flows Fp flow only rightward from the left of the side view, the flow rate increases, and the amount of decomposition processing increases.

On the other hand, in the case of hardly decomposable gas (for example, toluene), it is conceivable that the voltage is applied to the discharge electrodes 22a2, 22b2 of the gas decomposition elements 20e(2) to 20e(4), and the voltage is not applied to the discharge electrodes 22a1, 22b1. In this case, the plasma induced flows Fp flow leftward from the right of the side view, and similarly to the processing unit Uc, the residence time in its entirety becomes long and the decomposition efficiency increases.

In this manner, by choosing the direction of the plasma induced flows Fp according to the gas type and the gas concentration (for example, switching the alternate leftward and rightward flow directions by every stage), it becomes possible to change the flow path and the residence time, and the decomposition efficiency can be improved.

Moreover, by applying the voltage to the discharge electrodes 22a1, 22b1, 22a2, 22b2 to let the plasma induced flows Fp flow from the left and the right of the side view, the amount of generated OH radicals can be increased, and dirt on the discharge electrodes 22 can be cleaned efficiently.

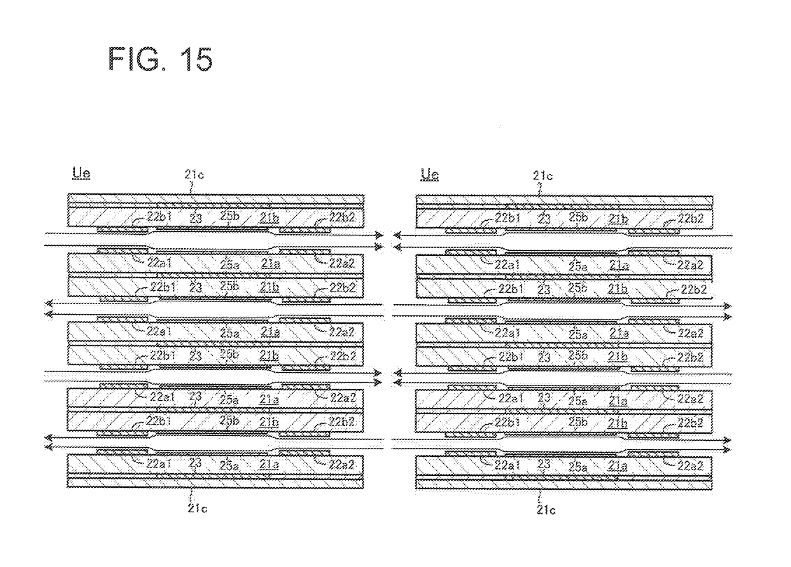

Further, as illustrated in FIG. 15, the two processing units Ue are disposed to oppose each other so as to let the gas to be processed flow vertically alternately and in reverse leftward and rightward, the flow rate of the gas to be processed decreases, and cleaning is carried out all over the discharge electrodes 22a, 22b.

At this time, the discharge electrodes 22a2, 22b2 over which the plasma induced flows Fp flow in the leftward direction (backward direction) of the side view when the voltage is applied are disposed on the downstream side of the gas decomposition elements 20e (stacks), and function as a flow limiter limiting the flow rate of the target gas.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A gas processing apparatus, comprising:
a plurality of stacks being away from each other and in parallel, each stack including a dielectric substrate, and a first to a third electrode, the dielectric substrate having a first and a second main surface, the first and second electrodes being respectively disposed on the first and second main surfaces, the third electrode being disposed inside the dielectric substrate;
a plurality of gas flow paths which supply a target gas between the stacks;
an AC power supply which apply an AC voltage across the first and second electrodes and the third electrodes, so as to generate a plurality of plasma induced flows of the target gas between the dielectric substrates; and
a flow limiter which limits a flow rate of the target gas, the flow limiter being disposed on a downstream side of the stacks.

2. The gas processing apparatus according to claim 1,
wherein a thickness of the gap between the dielectric substrates is equal to or less than 1.3 times a thickness of the plasma induced flow between the dielectric substrates,

3. The gas processing apparatus according to claim 1, further comprising an ozone treatment catalyst disposed between the stacks and the flow limiter,

4. The gas processing apparatus according to claim 3, further comprising a second ozone treatment catalyst disposed on an upstream side of the stacks.

5. The gas processing apparatus according to claim 1, further comprising a second flow limiter which limits a flow rate of the target gas, the second flow limiter disposed on an upstream side of the stacks.

6. The gas processing apparatus according to claim 1,
wherein the flow limiter includes:
a plurality of plate-shaped bodies away from each other; and
an adjuster which adjust an angle of the plate-shaped bodies.

7. The gas processing apparatus according to claim 1,
wherein the plasma-induced flows includes a first plasma-induced flow from upstream toward downstream and a second plasma-induced flow from downstream toward upstream.

8. The gas processing apparatus according to claim 7,
wherein the first and second plasma induced flows are disposed alternately,

9. The gas processing apparatus according to claim 7,
wherein electrodes among the first and second electrodes are disposed on an upstream side of the stacks and correspond to the first plasma-induced flow, and
wherein other electrodes among the first and second electrodes are disposed on a downstream side of the stacks and correspond to the second plasma-induced flow.

10. The gas processing apparatus according to claim 9, further comprising a guide which guides the first plasma-induced flow exited from the stacks toward the second plasma-induced flow.

11. The gas processing apparatus according to claim 1,
wherein the stacks each have fifth and sixth electrodes disposed corresponding to the first and second electrodes on the first and second main surfaces.

12. The gas processing apparatus according to claim 11, further comprising a switch which switches the first and second electrodes and the fifth and sixth electrodes and applying the AC voltage.

13. The gas processing apparatus according to claim 1, further comprising a controller which controls the flow limiter to periodically limit the flow rate of the target gas.

14. The gas processing apparatus according to claim 1, further comprising:
a dirt detector which detect dirt on the first and second electrodes; and
a controller which controls the flow limiter to limit the flow rate of the target gas according to a detection result by the dirt detector.

15. The gas processing apparatus according to claim 1, further comprising:
a gas detector which detects a type of gas; and
a controller which controls the flow limiter to limit the flow rate of the target gas according to a detection result by the gas detector.
